(19) United States
(12) Plant Patent Application Publication
(10) Pub. No.: US 2026/0114341 P1
(43) Pub. Date: Apr. 23, 2026

(54) Black Locust Tree Named 'OBE126'

(50) ;

(71) Applicant: Hungaroplant Kft, Budapest (HU)

(72) Inventor: Jeno Nemeth, Budapest (HU)

(21) Appl. No.: 19/363,191

(22) Filed: Oct. 20, 2025

Related U.S. Application Data
(60) Provisional application No. 63/709,692, filed on Oct. 21, 2024.

(51) Int. Cl. A01H 6/00; A01H 5/00

(52) U.S. Cl. CPC A01H 6/00; A01H 5/00
USPC PLT/216

(57) ABSTRACT

A new black locust tree ‘OBE126’ particularly distinguished by ovate leaves with an emarginate apex; lance like thorns; straight, pinnate growth habit; and blooms profusely and is a useful bee pasture, well-liked by beekeepers, is disclosed.

### CROSS REFERENCE TO RELATED MATTER

The present application claims priority to U.S. Application No. 63/709,692, as filed on Oct. 21, 2024, the entire contents of which is incorporated herein by reference for all purposes.

Genus and species: Robinia pseudoacacia.

Variety denomination: ‘OBE126’

### BACKGROUND OF THE NEW PLANT ‘OBE126’

The present invention comprises a new and distinct variety of black locust tree, botanically known as Robinia pseudoacacia and hereinafter referred to by the variety name ‘OBE126’. ‘OBE126’ was selected from experimental Black locust (Robinia pseudoacacia) plantation established on the Great Hungarian Plain near town Nagykőrös, in 2020.

‘OBE126’ was asexually reproduced in 2020 by vegetative cuttings from budwood.

### SUMMARY OF ‘OBE126’

The following are the most outstanding and distinguishing characteristics of this new variety when grown under normal horticultural practices near Nagykörös, Hungary.
1. Ovate leaves with an emarginate apex;
2. Lance like thorns;
3. Straight, pinnate growth habit; and
4. Blooms profusely and is a useful bee pasture, well-liked by beekeepers.

### DESCRIPTION OF THE PHOTOGRAPHS

These new Black Locust trees are illustrated by the accompanying photographs of plants grown near Nagykörös, Hungary. The colors shown are as true as can be reasonably obtained by conventional photographic procedures.

FIG. 1 shows the overall shape of the new ‘OBE126’ variety.

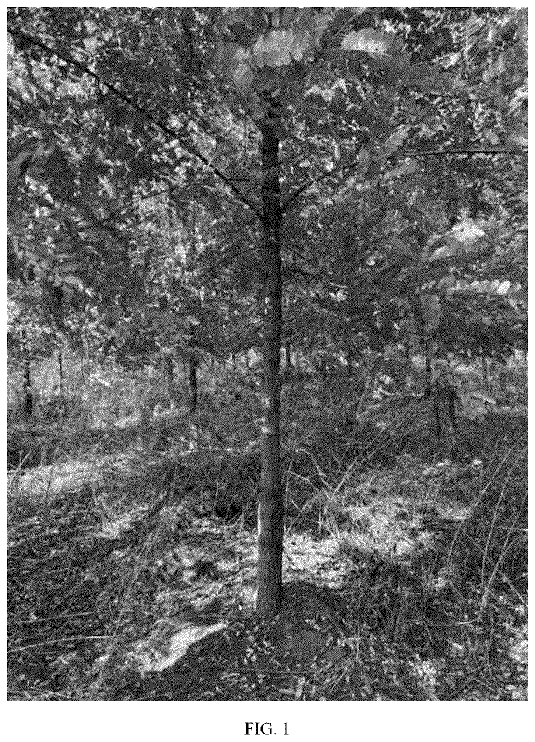

FIG. 2 shows a close-up of the bark and thorns of ‘OBE126’.

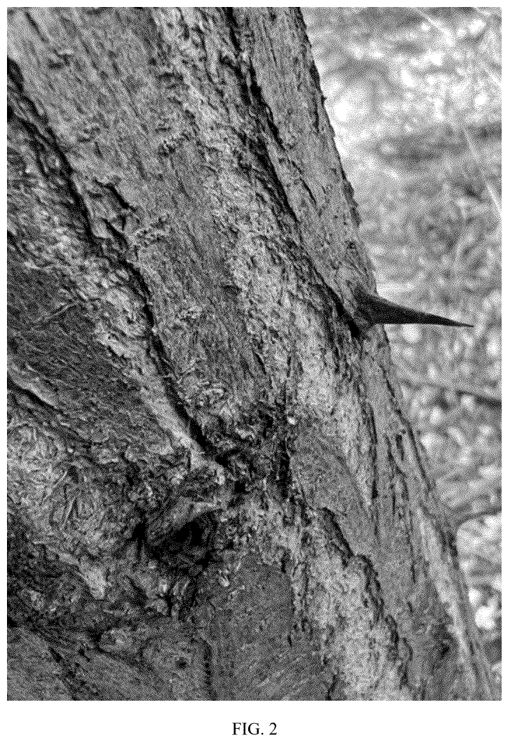

FIG. 3 shows a close-up view of the leaves of ‘OBE126’.

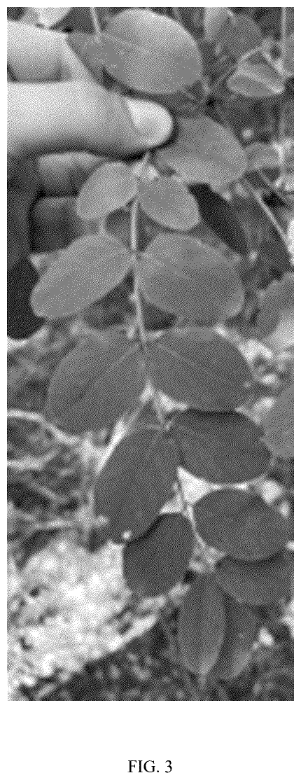

### DESCRIPTION OF THE ‘OBE126’ VARIETY

The following detailed descriptions set forth the distinctive characteristics of ‘OBE126’. The data which define these characteristics was collected near Nagykörös, Hungary. Color references are to The R.H.S. Colour Chart of The Royal Horticultural Society of London (R.H.S.), 6th edition (2016).
Classification:
   Family.—Fabaceae.
   Botanical.—Robinia pseudoacacia.
   Common name.—Black locust.
   Variety name.—‘OBE126’.
Parentage:
   Female parent.—Unnamed Experimental black locust.
   Male parent.—Unknown.
General description:
   Plant habit.—Deciduous tree with straight, upright growth habit. At the age of three, it is a small tree with a diameter of 3.0 to 7.0 cm and a height of 5.0 to 8.0 m.
   Bark color (mature stems).—Predominately Greyed-Brown Group 199B; ridges and older, shaded fissures approach Greyed-Brown Group 199A to Black Group 202A; freshly exfoliated/abraded areas show warm tones near Greyed-Orange Group 165B-165C.
   Crotch angles.—60-70.
   Smaller branches in 1 m.—Average of 7.66.
   Larger branches in 1 m.—Average of 2.
   Stem straightness.—no curves, straight stem.
   USDA hardiness zone.—Zone 4.
   Thorns.—Lance like.
   Diseases and pest resistance.—Resistant to Locust borer (Megacyllene robiniae), Locust leaf miner (Odontota dorsalis)
Foliage description:
   Color.—Medium green.
   Variegation.—Absent.
   Leaf shape.—Middle section of leaf: Elliptic. Apex: Emarginate.
Flowers description:
   Conspicuousness.—Medium.
   Type.—Single.
   Color.—White.
Fruit: Absent.

I claim:

1. A new and distinct variety of Black Locust tree named ‘OBE126’ as shown and described herein.
